(19) United States
(12) Patent Application Publication
(10) Pub. No.: US 2026/0315724 A1
(43) Pub. Date: Oct. 15, 2026

(54) HYPOXIA DEVICE AND METHOD FOR CARRYING OUT HYPOXIA TRAINING

(71) Applicant: Egor EGOROV, Berlin (DE)

(72) Inventor: Egor EGOROV, Berlin (DE)

(21) Appl. No.: 19/485,595

(22) PCT Filed: May 17, 2024
(86) PCT No.: PCT/EP2024/063655

§ 371 Date: Nov. 18, 2025

(30) Foreign Application Priority Data
May 19, 2023 (DE) 10 2023 113 214.8

(51) Int. Cl. A61M 16/12; A61M 16/00; A61M 16/10; A61M 16/20; A61M 16/22

(52) U.S. Cl. CPC A61M 16/125; A61M 16/0051; A61M 16/202; A61M 16/22; A61M 2016/103

(57) ABSTRACT

The invention relates to a hypoxia device for carrying out a hypoxia treatment and/or hypoxia training, said device having a gas reservoir, a gas dispensing device, a supply line that is suitable and intended for conducting a gas mixture from the gas reservoir to the gas dispensing device, and/or a discharge line that is suitable and intended for conducting a gas mixture from the gas dispensing device to the gas reservoir, having a valve and/or sensor, wherein the valve and/or sensor is arranged in the supply line, the gas reservoir, and/or the gas dispensing device, and having an automatic switch-off device. The invention also relates to a method for carrying out a hypoxia treatment and/or a hypoxia training, having the method steps of supplying and/or discharging a gas mixture from a gas reservoir via a supply line and/or discharge line and a CO₂ absorber arranged in the supply line and/or discharge line, to a gas dispensing device; monitoring the use of the CO₂ absorber and controlling an automatic switch-off device depending on the use of the CO₂ absorber.

The invention relates to a hypoxia device for carrying out a hypoxia treatment and/or hypoxia training, said device having a gas reservoir, a gas dispensing device, a supply line that is suitable and intended for conducting a gas mixture from the gas reservoir to the gas dispensing device, and/or a discharge line that is suitable and intended for conducting a gas mixture from the gas dispensing device to the gas reservoir, having a valve and/or sensor, wherein the valve and/or sensor is arranged in the supply line, the gas reservoir, and/or the gas dispensing device, and having an automatic switch-off device. The invention also relates to a method for carrying out a hypoxia treatment and/or a hypoxia training, having the method steps of supplying and/or discharging a gas mixture from a gas reservoir via a supply line and/or discharge line and a CO₂ absorber arranged in the supply line and/or discharge line, to a gas dispensing device; monitoring the use of the CO₂ absorber and controlling an automatic switch-off device depending on the use of the CO₂ absorber.

### PRIOR ART

Hypoxia can cause reactions in every body cell and can enable an increased energy metabolism. It can contribute to the activation of a variety of genes. Athletes, healthy people, and the sick may benefit from hypoxia.

It has been known for a long time that altitude training has beneficial effects. But how the slight lack of oxygen leads to an improvement in performance in the body could not be explained sufficiently until a few years ago. The observed increase in red blood cells was not sufficient to explain the changes in the body. The breakthrough for understanding came with the discovery of the hypoxia-inducible factor HIF-1-alpha. It provided the explanation for the comprehensive effect of altitude training. The abbreviation HIF stands for Hypoxia-Inducible Factor. Behind the technical term lies an oxygen sensor, which becomes active when there is not enough oxygen in the body cells. It controls one of the most important processes in the body for survival: the adaptation of cells, tissues and organs to a lack of oxygen. At the same time, it is a signal for self-repair in the body.

The best known positive effect of HIF is erythropoietin synthesis (EPO) in the kidney and liver. It served to explain the changes in the cardiovascular, breathing and blood systems before the discovery of HIF. It is now clear that the performance improvement is much more comprehensive. The endothelial cells of the intimal tunica react to the impact of hypoxia with an increased nitric oxide synthesis (NO). The gas has a decisive impact on the dilatation of the vessels. It leaves the endothelium and has a relaxing effect on smooth muscle cells in the surrounding tissue. On the intimal tunica itself, NO prevents platelet adhesion and aggregation. It is also interesting in this context that the endothelial cells form the angiogenetic factor VEGF under the impact of hypoxia. As a result of its formation, neoangiogenesis of the capillary vessels occurs in the context of hypoxia therapy. Very often, these additional vessels are located in damaged or poorly perfused tissue sections.

The heart beats faster and people breathe more often. The body tries to absorb as much oxygen as possible from the air it breathes. For humans, this is neither unpleasant nor dangerous. The vessels widen, and blood can flow faster. The number of red blood cells is elevated, and the body can absorb more oxygen molecules. Supply to all organs is improved. In addition, the body starts an adjustment program. Under the impact of hypoxia factor HIF-1-alpha, the body takes all precautions to get by even with less oxygen.

The mitochondria become dense and rejuvenated. The energy production is optimized, which leads to more physical and mental performance in everyday life. In people with disorders, symptoms lessen. In the brain, the transmission of nerve impulses improves. In some areas, new nerve cells develop. Consequently, mental strength increases and the risk of dementia and Parkinson's disease is lowered. The vascular network becomes denser. The inner walls of the vessels become smooth and supple. This reduces the chance of dangerous blood clots, as well as the risk of heart attack and stroke.

On the other hand, lack of oxygen can lead to inability to concentrate, fatigue, and eventually loss of consciousness, causing significant damage. An optimal training program must therefore take into account the personal limits of each person to be trained in order to be safe for the subject, but should also be in an area in which the organism can be optimally addressed and the training is effective.

It is therefore an object of the present invention to provide a method for carrying out hypoxia training with a hypoxia device, which enables a safe and at the same time efficient execution of hypoxia training. It is likewise an object of the present invention to provide a hypoxia device for carrying out a hypoxia treatment and/or hypoxia training, which enables safe and at the same time efficient carrying out of hypoxia training.

The stated object is achieved by means of the hypoxia device for carrying out a hypoxia treatment and/or a hypoxia training according to claim 1. Further advantageous configurations of the invention are set out in the dependent claims.

The device according to the invention for carrying out a hypoxia treatment and/or hypoxia training comprises a gas reservoir and a gas dispensing device. The gas reservoir is an air reservoir that has the gas mixture for carrying out a hypoxia treatment and/or hypoxia training. The gas dispensing device is usually configured as a breathing mask that a user wears over his or her breathing openings (mouth and nose) to carry out a hypoxia treatment and/or hypoxia training.

Furthermore, the hypoxia device has a supply line that is suitable and intended to conduct a gas mixture from the gas reservoir to the gas dispensing device and/or a discharge line that is suitable and intended to conduct a gas mixture from the gas dispensing device to the gas reservoir. The supply and/or discharge line is usually configured as a flexible hose and connects the gas dispensing device to the gas reservoir in a gas-tight manner.

Furthermore, the hypoxia device comprises an automatic shut-off device. By means of the automatic shut-off device, hypoxia treatment and/or hypoxia training can be automatically interrupted if the CO₂ content of the respiratory gas in the gas reservoir is so high that there is a health risk for the user, for example. By means of the hypoxia device according to the invention, an effective hypoxia treatment and/or hypoxia training is therefore possible; all adaptation processes are initiated in the user's body, but no physical damage is caused by the intentionally induced oxygen deficiency.

In a further advantageous development of the invention, the gas supply to the gas dispensing device can be interrupted by the automatic shut-off device. In particular, the gas flow can be automatically interrupted before a user suffers damage due to a lack of oxygen or increased CO₂ concentration.

In an advantageous embodiment of the invention, the hypoxia device for carrying out a hypoxia treatment comprises a CO₂ absorber. The CO₂ absorber is preferably arranged in the breathing circuit in the supply or discharge line. The CO₂ absorber is usually a mixture of calcium hydroxide and sodium hydroxide in solid form (so-called soda lime). During hypoxia treatment and/or hypoxia training, the air flows through the soda lime in which the carbon dioxide is first bound to sodium hydroxide, which is then regenerated by the calcium hydroxide, which is also contained therein.

In a further embodiment of the invention, the CO₂ absorber is replaceable. In a further development of the invention, the CO₂ absorber is arranged in a flow-through container. The CO₂ absorber is consumed during hypoxia treatment and/or hypoxia training and therefore usually needs to be replaced before or after each hypoxia treatment and/or hypoxia training or after consumption of the CO₂ absorber. For this purpose, the hypoxia device according to the invention has a flow-through container such that the CO₂ absorber is replaceable. The flow-through container is located in the supply or discharge line, and the gas mixture flows therethrough for carrying out the hypoxia treatment and/or hypoxia training.

In a further embodiment of the invention, the flow-through container has an inlet and/or outlet, wherein the flow-through container can be closed so that the flow of gas through the flow-through container is prevented. The inlet and outlet ensure that the gas mixture for carrying out the hypoxia treatment and/or hypoxia training flows through the flow-through container. In a further development of the invention, the flow-through container has an inlet and/or outlet, wherein the inlet and/or outlet can be closed so that the flow of gas through the flow-through container is prevented. In a further aspect of the invention, the supply line and/or the discharge line can be closed so that the flow of gas through the flow-through container is prevented. The flow-through container can be closed such that the flow of the gas mixture through the flow-through container is interrupted. In addition, the flow-through container is designed to be openable to allow the gas mixture to flow again.

In a further embodiment of the invention, the automatic shut-off device is coupled to a controller, wherein the automatic shut-off device can be controlled by the controller. The controller includes a programmable microprocessor and a storage device with a suitable software program. The controller sends a control signal to the automatic shut-off device if the CO₂ content of the respiratory gas in the gas reservoir is so high that there is a health risk for the user, for example.

In a further embodiment of the invention, the use of the CO₂ absorber can be monitored by the controller. The CO₂ absorber is usually a mixture of calcium hydroxide and sodium hydroxide in solid form (so-called soda lime). During hypoxia treatment and/or hypoxia training, the air flows through the soda lime, in which the carbon dioxide is first bound to sodium hydroxide, which is then regenerated by the calcium hydroxide, also known as slaked lime, which is also contained therein. The CO₂ absorber is consumed through its use during hypoxia treatment and/or hypoxia training. The use of the CO₂ absorber can be determined, for example, by the duration of a hypoxia treatment and/or hypoxia training, or by detecting a CO₂ concentration in the respiratory gas.

In a further embodiment of the invention, the use of the CO₂ absorber can be monitored by counting the number of hypoxia treatments. In a further development of the invention, the use of the CO₂ absorber can be monitored by determining the time of use of the CO₂ absorber. The use of the CO₂ absorber can be monitored via the duration of a hypoxia treatment and the usage cycles.

In a further embodiment of the invention, the hypoxia device comprises a flow sensor, wherein the use of the CO₂ absorber can be monitored by determining the gas flow through the CO₂ absorber by means of the flow sensor. The volume of respiratory gas flowing through the CO₂ absorber is a measure of the ability of the CO₂ absorber to absorb carbon dioxide.

In a further embodiment of the invention, the hypoxia device for carrying out a hypoxia treatment comprises a CO₂ sensor, wherein the monitoring of the use of the CO₂ absorber can be monitored by a CO₂ sensor. The CO₂ sensor determines the carbon dioxide concentration in the respiratory gas. The CO₂ content of the air inhaled and exhaled by a user through the gas dispensing device is preferably continuously detectable.

In a further embodiment of the invention, the automatic shut-off device comprises a valve through which the gas flow from the gas reservoir to the gas dispensing device can be interrupted. If the determined CO₂ concentration is exceeded, the shut-off device interrupts the gas supply to the gas dispensing device such that a user can no longer carry out the hypoxia training and must remove the gas dispensing device from his or her breathing openings. In a further embodiment of the invention, the shut-off device comprises a shut-off valve which is arranged in the supply line and/or discharge line, for example. In a further development of the invention, the automatic shut-off device comprises a blocking element through which the gas flow from the gas reservoir to the gas dispensing device can be interrupted by rendering the CO₂ absorber unusable.

The object is furthermore achieved by means of the method according to the invention for carrying out a hypoxia treatment and/or hypoxia training. Further advantageous embodiments of the invention are also set forth in the dependent claims.

The method according to the invention for carrying out a hypoxia treatment and/or hypoxia training, comprises three method steps: The first method step involves supplying and/or discharging a gas mixture from a gas reservoir via a supply line and/or discharge line and a CO₂ absorber arranged in the supply line and/or discharge line, to a gas dispensing device. The gas mixture has, in particular, a reduced oxygen content compared to conventional breathing air. The second method step involves monitoring the use of the CO₂ absorber. The usage, e.g., the duration of use of the CO₂ absorber, is a measure of the ability of the CO₂ absorber to absorb carbon dioxide.

In the third method step, an automatic shut-off device is controlled depending on the use of the CO₂ absorber. The automatic shut-off device is preferably activated when the detected usage exceeds a limit. The automatic shut-off device is automated in such a way that the automatic shut-off device is activated without user involvement. In the simplest case, the automatic shut-off device interrupts the gas supply to the gas dispensing device such that a user can no longer carry out the hypoxia treatment and/or hypoxia training. Alternatively or additionally, the automatic shut-off device is controlled such that an optical and/or acoustic warning signal is generated.

In a further embodiment of the invention, monitoring the use of the CO₂ absorber comprises monitoring the consumption of the CO₂ absorber. When the CO₂ absorber is used up, it can no longer absorb carbon dioxide from the gas mixture. Monitoring the consumption of the CO₂ absorber is therefore necessary to enable safe hypoxia treatment and/or hypoxia training for a user.

In a further embodiment of the invention, controlling the automatic shut-off device comprises controlling a blocking element. If the determined use of the CO₂ absorber is exceeded, the automatic shut-off device interrupts and/or reduces the gas supply to the gas dispensing device such that a user can no longer carry out the hypoxia training and must remove the gas dispensing device from his or her breathing openings. For this purpose, a shut-off valve is activated and the blocking element changes the gas supply to the gas dispensing device.

In a further design of the invention, the blocking element comprises a controllable valve and/or a sealable flow-through container. The controllable valve can be used to interrupt and/or reduce the gas supply to the gas dispensing device. The sealable flow-through container comprises a device for interrupting the gas supply to the gas dispensing device.

In a further development of the invention, the blocking element is controlled to interrupt and/or change the gas flow through the hypoxia device in order to carry out a hypoxia treatment and/or a hypoxia training. During the hypoxia treatment and/or hypoxia training, exhaled carbon dioxide accumulates in the gas mixture of the breathing circuit. There are also physiological risks associated with too much carbon dioxide in the inhaled air. When the CO₂ absorber is used up, it can no longer absorb carbon dioxide from the gas mixture. Controlling the blocking element to interrupt and/or change the gas flow is therefore necessary to enable a safe hypoxia treatment and/or hypoxia training for a user.

In a further implementation of the invention, controlling the blocking element results in supplying ambient air into the hypoxia device for carrying out a hypoxia treatment and/or hypoxia training. By supplying ambient air into the hypoxia device, the CO₂ concentration in the hypoxia device is reduced to such an extent that the danger to a user is reduced.

In a further design of the invention, the CO₂ absorber is arranged in a flow-through container. The CO₂ absorber is consumed during the hypoxia treatment and/or hypoxia training and therefore usually needs to be replaced before or after each hypoxia treatment and/or hypoxia training or after it has been used up. The flow-through container contains the CO₂ absorber and is arranged in the breathing circuit of the hypoxia device according to the invention. The flow-through container holds the CO₂ absorber, wherein the flow-through container is configured as an exchangeable cartridge that can be replaced with an unused cartridge before or after the hypoxia treatment and/or hypoxia training.

In a further embodiment of the invention, the CO₂ absorber is arranged in a flow-through container, wherein the flow-through container has an inlet and/or outlet, wherein the flow-through container can be closed so that the flow of gas through the flow-through container is prevented. The gas mixture for carrying out the hypoxia treatment and/or hypoxia training can be conducted through the flow-through container. Therefore, the container is arranged in the supply and/or discharge line and the CO₂ absorber arranged in the container therefore only absorbs carbon dioxide from the user's breathing air and is not contaminated by carbon dioxide from the ambient air. This ensures that the CO₂ absorber arranged in the container removes the carbon dioxide from the gas mixture. In a further development of the invention, the inlet and/or the outlet can be closed. The inlet and/or outlet can be closed by the automatic shut-off device and interrupt the gas supply to the gas dispensing device such that a user can no longer carry out the hypoxia treatment and/or hypoxia training and must remove the gas dispensing device from his or her breathing openings.

In a further embodiment of the invention, the gas flow through the flow-through container is prevented by a gas-impermeable substance. For this purpose, the flow-through container preferably has a valve that can be closed and opened.

In an advantageous embodiment of the invention, the benefit of the CO₂ absorber is monitored by measuring the CO₂ concentration in the hypoxia device for carrying out a hypoxia treatment. For this purpose, the hypoxia device according to the invention has a CO₂ sensor which is arranged in the supply line or the discharge line of the breathing circuit. The CO₂ content of the breathing air can preferably be measured continuously using the CO₂ sensor. The CO₂ sensor may, for example, comprise an optical and/or acoustic display and/or be connected to an optical and/or acoustic display that emits a warning signal when the CO₂ content of the breathing air in the breathing circuit detected by the CO₂ sensor reaches a value that is dangerous for a user.

In a further advantageous design of the invention, the use of the CO₂ absorber is monitored with a CO₂ sensor by recording a measured value. The CO₂ content of the breathing air that a user inhales and exhales through the gas dispensing device can preferably be monitored continuously by detecting measurement values that are detected at similar time intervals.

In a further development of the invention, the CO₂ sensor is arranged in the gas reservoir, the gas dispensing device, the supply line, and/or the discharge line. The CO₂ sensor is located in the breathing circuit of the hypoxia device and detects the CO₂ concentration of the hypoxic gas mixture during hypoxia treatment and/or hypoxia training.

In a further embodiment of the invention, the measured value determined by the CO₂ sensor is compared to a previously defined threshold value. The controller of the hypoxia device has a threshold value of the CO₂ concentration of the gas delivered via the gas dispensing device. If the CO₂ content determined by the CO₂ sensor exceeds the threshold value, a corresponding signal is output and/or the gas delivery through the gas dispensing device is stopped.

In a further development of the invention, a first threshold value is fixed and/or a second threshold value is variably set. The first threshold value is preferably adjustable such that when the CO₂ concentration determined by the CO₂ sensor is exceeded, the automatic shut-off device blocks the gas supply to the gas dispensing device. The second threshold value can be adjusted to provide an acoustic and/or visual warning to the user, but the user can continue the hypoxia treatment and/or hypoxia training. The first threshold value therefore is at a higher CO₂ concentration than the second threshold value.

In a further embodiment of the invention, a warning is output when the threshold value is reached and/or exceeded. The output is acoustic and/or visual.

In a further development of the invention, the warning is output before the blocking element is controlled to interrupt and/or change the gas flow in the hypoxia device for carrying out a hypoxia treatment and/or hypoxia training. The warning is output when the measured CO₂ concentration exceeds the second threshold value. The gas flow is interrupted when the determined CO₂ concentration exceeds the first threshold value, wherein the first threshold value is at a higher CO₂ concentration of the gas mixture than the second threshold value. Typically, the second threshold value is exceeded earlier than the first threshold value. This means that first a warning is therefore given before the gas flow is interrupted.

Exemplary embodiments of the hypoxia device according to the invention for carrying out a hypoxia treatment and/or hypoxia training and of the method according to the invention for carrying out hypoxia training are schematically shown in the figures in a simplified manner and are explained in more detail in the following description.

In the figures:

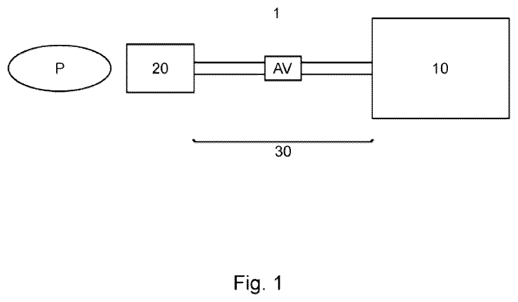

FIG. 1: shows the hypoxia device according to the invention for carrying out a hypoxia treatment and/or hypoxia training, a supply line and a discharge line

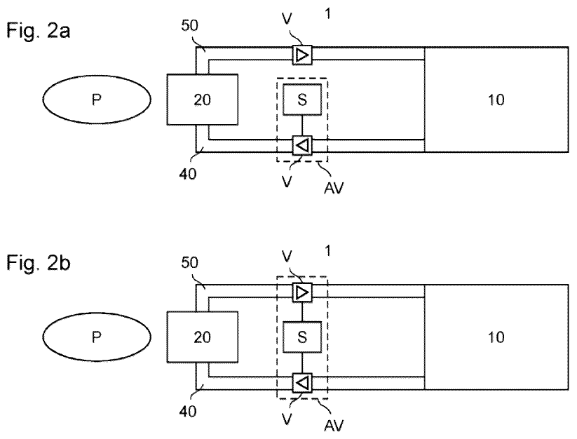

FIG. 2a: shows the hypoxia device according to the invention for carrying out a hypoxia treatment and/or hypoxia training, a supply line and a discharge line separately, an automatic shut-off device arranged in the supply line

FIG. 2b: shows the hypoxia device according to the invention for carrying out a hypoxia treatment and/or hypoxia training, a supply line and a discharge line separately, an automatic shut-off device arranged in the discharge line

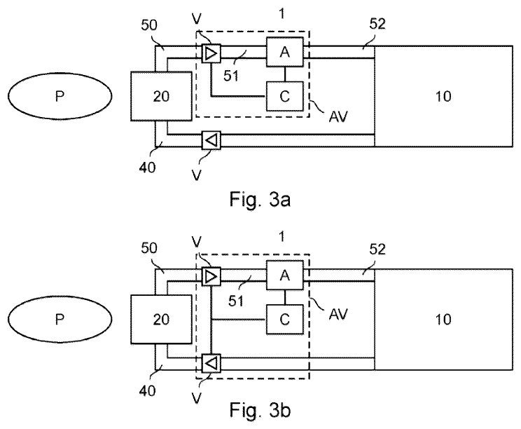

FIG. 3a: shows the hypoxia device according to the invention for carrying out a hypoxia treatment and/or hypoxia training, a supply line and a discharge line separately, an automatic shut-off device arranged in the discharge line

FIG. 3b: shows the hypoxia device according to the invention for carrying out a hypoxia treatment and/or hypoxia training, a supply line and a discharge line separately, an automatic shut-off device arranged in the supply line and the discharge line

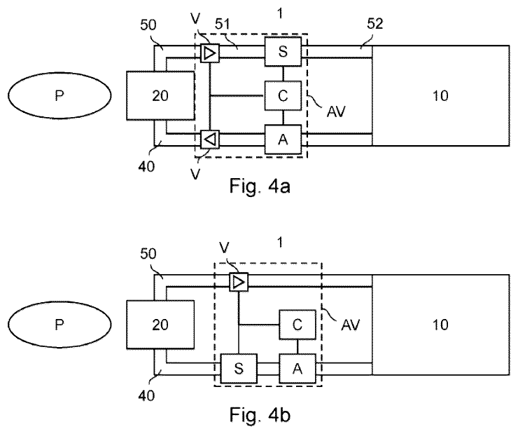

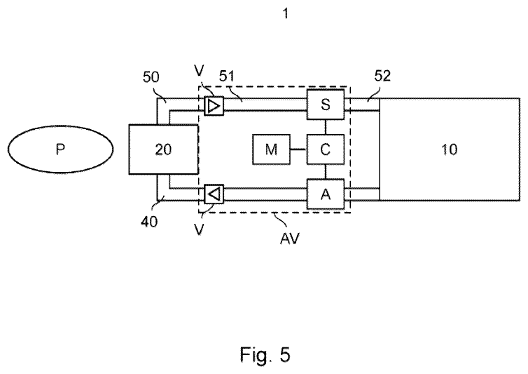

FIG. 4a: shows the hypoxia device according to the invention for carrying out a hypoxia treatment and/or hypoxia training, a supply line and a discharge line separately, an automatic shut-off device arranged in the supply line and the discharge line, a sensor arranged in the discharge line

FIG. 4b: shows the hypoxia device according to the invention for carrying out a hypoxia treatment and/or hypoxia training, a supply line and a discharge line separately, an automatic shut-off device arranged in the supply line and the discharge line, a sensor arranged in the supply line

FIG. 1 shows an exemplary embodiment of the hypoxia device 1 according to the invention for carrying out a hypoxia treatment and/or hypoxia training. The hypoxia device 1 comprises the gas dispensing device 20, which is configured as a breathing mask and is worn by the user P over his or her breathing openings (mouth and nose) during the hypoxia treatment and/or hypoxia training. The hypoxia device 1 also comprises the gas reservoir 10. The gas reservoir 10 and the gas dispensing device 20 are connected to each other in a gas-tight manner via the flexible gas line 30. The gas line 30 has the automatic shut-off device AV for changing the gas flow of the respiratory gas between the gas reservoir 10 and the gas dispensing device 20. The automatic shut-off device AV is arranged in a flow-through container in the gas line 30 such that the gas mixture of the respiratory gas is conducted through the flow-through container and the shut-off device AV during the hypoxia treatment and/or hypoxia training.

FIG. 2 shows a further embodiment of the hypoxia device 1 according to the invention for carrying out a hypoxia treatment and/or hypoxia training. The hypoxia device 1 also comprises the gas dispensing device 20. The gas dispensing device 20 is connected to the gas reservoir 10 via a flexible supply line 40 and a likewise flexible discharge line 50. The supply line 40 and the discharge line 50 each comprise a gas-tight one-way valve V. One-way valves V ensure that user P only inhales the air from the supply line 40 and thus prevent user P from inhaling CO₂-containing air from the discharge line 50 when inhaling. However, the CO₂ sensor S can also be arranged in the gas reservoir 10 or in the gas dispensing device 20. The automatic shut-off device AV is arranged in a flow-through container in the supply line 40 (FIG. 2a). The automatic shut-off device AV comprises the sensor S, which permanently detects the CO₂ concentration of the respiratory gas in the supply line 40 while carrying out a hypoxia treatment and/or hypoxia training.

The CO₂ sensor S is coupled to the one-way valve V in the supply line 40. If an adjustable threshold value of the CO₂ concentration of the respiratory gas is exceeded, the CO₂ sensor S sends a signal to the one-way valve V in the supply line 40. The threshold value for the CO₂ concentration of the respiratory gas is 3% of the respiratory gas in all exemplary embodiments. The signal contains a command such that the one-way valve V in the supply line 40 closes, thus preventing the flow of the respiratory gas through the flow-through container.

In an alternative embodiment, the automatic shut-off device AV is arranged in a flow-through container in the discharge line 50 (FIG. 2b), the CO₂ sensor S is coupled to the one-way valve V in the discharge line 50. If an adjustable threshold value of the CO₂ concentration of the respiratory gas is exceeded, the CO₂ sensor S sends a signal to the one-way valve V in the discharge line 50. The signal contains a command such that the one-way valve V in the discharge line 50 closes, the gas flow of the respiratory gas through the flow-through container is prevented as well.

Alternatively, the flow-through container has a closable inlet and/or a likewise closable outlet, wherein the flow-through container can be closed by means of a one-way valve in the inlet and/or outlet so that the flow of gas through the flow-through container is prevented.

In a further embodiment, the sensor S is a flow sensor which detects the volume of respiratory gas flowing through the supply line 40 or discharge line 50 and generates a corresponding measurement signal to the controller S. If an adjustable threshold value of the volume of the respiratory gas is exceeded, the sensor S sends a signal with the command to close the one-way valve in the supply line 40 and/or the discharge line 50.

FIG. 3 shows a further exemplary embodiment of the hypoxia device 1 according to the invention. The hypoxia device 1 also has a separate supply line 40 and a discharge line 50, each with a one-way valve V. The hypoxia device 1 comprises the automatic shut-off device AV in which the controller C and the CO₂ absorber A are arranged. The CO₂ absorber A is arranged in a flow-through container in the discharge line 50 between the first region 51 and the second region 52 of the discharge line 50 and is designed to be replaceable. In all exemplary embodiments shown here, the CO₂ absorber A is a mixture of calcium hydroxide Ca(OH)₂ and sodium hydroxide NaOH, also referred to as soda lime. The CO₂ absorber has a limited capacity to absorb carbon dioxide, so it is consumed during hypoxia treatment and/or hypoxia training when the CO₂ absorber is exposed to carbon dioxide from the respiratory air. The controller C is connected to the CO₂ absorber A and the valve V in the discharge line 50 (FIG. 3a), in a variant the controller C is connected to the CO₂ absorber A, the one-way valve V in the discharge line 50 and the one-way valve V in the supply line 40 (FIG. 3b).

To carry out a hypoxia treatment and/or hypoxia training, a user P places the gas dispensing device 20 over his or her breathing openings and repeatedly inhales and exhales the same respiratory gas. The CO₂ absorber A prevents suffocation by first binding the carbon dioxide to sodium hydroxide, which is then regenerated by the calcium hydroxide it also contains. The controller C monitors the use of the CO₂ absorber A by detecting the time of use of the CO₂ absorber A. The time of use is a measure of the exposure of the CO₂ absorber A to carbon dioxide. The controller C compares this determined time of use of the CO₂ absorber A with a value for the time of use of the CO₂ absorber A stored in the memory unit of the controller C. If the determined time of use of the CO₂ absorber A reaches or exceeds the stored value for the time of use of the CO₂ absorber A, the controller C sends a control signal to the one-way valve V in the outlet line 50 (FIG. 3a) or to both valves V in both the outlet line 50 and the supply line 40 (FIG. 3b), such that the one-way valve V or the one-way valves V interrupt the gas supply to the gas dispensing device 20. To do this, the one-way valve V or the one-way valves V are closed.

FIG. 4 shows a preferred exemplary embodiment of the hypoxia device 1 according to the invention. The hypoxia device 1 also comprises the gas dispensing device 20. The gas dispensing device 20 is connected to the gas reservoir 10 via a flexible supply line 40 and a likewise flexible discharge line 50. The supply line 40 and the discharge line 50 each have a one-way valve V. The hypoxia device 1 comprises the automatic shut-off device AV in which the controller C, the sensor S, and the CO₂ absorber A are arranged. The CO₂ absorber A is arranged in a flow-through container in the supply line 40 and is designed to be replaceable. The sensor S is arranged in the discharge line 50 between the first region 51 and the second region 52 of the discharge line 50 (FIG. 4a). The controller C is connected to the two one-way valves V, the CO₂ absorber, and the sensor S. In an alternative variant of this exemplary embodiment, the sensor S is arranged in the supply line 40 (FIG. 4b) and connected to the controller C. The supply line 40 does not have a one-way valve V, the outlet line 50 has a one-way valve V as well as the sensor S. The controller C is connected to the one-way valve V, the CO₂ absorber, and the sensor S. The sensor S in this exemplary embodiment is a CO₂ sensor which permanently detects the CO₂ concentration of the respiratory gas while carrying out a hypoxia treatment and/or hypoxia training.

To carry out the hypoxia treatment and/or hypoxia training, a user P places the gas dispensing device 20 over his or her breathing openings and repeatedly inhales and exhales the same respiratory gas. The CO₂ absorber A prevents the user P from suffocating. The sensor monitors the use of the CO₂ absorber A in such a way that the sensor continuously detects measured values of the CO₂ concentration of the respiratory gas. The measured values are sent from the sensor S to the controller C, where they are compared with a first threshold value for the CO₂ concentration of the respiratory gas. The first threshold value is stored in the memory unit of the controller and is fixed. If the measured CO₂ concentration of the respiratory gas reaches or exceeds the first threshold value, the controller C outputs an acoustic warning. For this purpose, the controller V is equipped with a suitable loudspeaker or connected to one. The threshold value for the CO₂ concentration of the respiratory gas is 3% of the respiratory gas.

A second threshold value which can be variably set, for example, by the user P or a doctor, is stored in the memory unit of the controller C. The second threshold value for the CO₂ concentration of the respiratory gas is 5% of the respiratory gas. If the measured CO₂ concentration of the respiratory gas reaches or exceeds the second threshold value, the controller sends a control command to the one-way valve V in the supply line 40 such that the one-way valve V in the supply line 40 is closed (FIG. 4a) and thus interrupts the gas flow of the respiratory gas in the supply line 40, the carrying out of a hypoxia treatment and/or a hypoxia training is interrupted. The acoustic warning when the first threshold value of the measured CO₂ concentration of the respiratory gas is reached and/or exceeded thus occurs before the one-way valve V in the supply line 40 is controlled to interrupt the gas flow in the hypoxia device 1 when the second threshold value of the measured CO₂ concentration of the respiratory gas is reached and/or exceeded.

In the variant of the exemplary embodiment (FIG. 4b), the one-way valves V in both the supply line 40 and the discharge line 50 are closed when the second threshold value of the measured CO₂ concentration of the respiratory gas is reached and/or exceeded.

LIST OF REFERENCE NUMERALS

| | |
|---|---|
| 1 | hypoxia device for carrying out a hypoxia treatment and/or |
| | hypoxia training |
| 10 | gas reservoir |
| 20 | gas dispensing device / breathing mask |
| 30 | gas line |
| 40 | supply line |
| 50 | discharge line |
| 51 | first region of the discharge line |
| 52 | second region of the discharge line |
| A | CO₂ absorber |
| AV | automatic shut-off device |
| C | controller |
| P | user |
| S | sensor |
| V | one-way valve |

1. A hypoxia device (1) for carrying out a hypoxia treatment and/or hypoxia training, having:
a gas reservoir (10),
a gas dispensing device (20),
a supply line (40) that is suitable and intended for conducting a gas mixture from the gas reservoir (10) to the gas dispensing device (20) and/or a discharge line (50) that is suitable and intended for conducting a gas mixture from the gas dispensing device (20) to the gas reservoir (10), and an
automatic shut-off device (AV).

2. The hypoxia device (1) for carrying out a hypoxia treatment and/or hypoxia training of claim 1,
characterized in that
the gas supply from the gas reservoir (10) to the gas dispensing device (20) can be interrupted by the automatic shut-off device (AV).

3. The hypoxia device (1) for carrying out a hypoxia treatment and/or hypoxia training of claim 1,
characterized in that
the hypoxia device (1) for carrying out a hypoxia treatment comprises a CO₂ absorber (A).

4. The hypoxia device (1) for carrying out a hypoxia treatment and/or hypoxia training of claim 3,
characterized in that
the CO₂ absorber (A) is replaceable.

5. The hypoxia device (1) for carrying out a hypoxia treatment and/or hypoxia training of claim 3,
characterized in that
the CO₂ absorber (A) is arranged in a flow-through container.

6. The hypoxia device (1) for carrying out a hypoxia treatment and/or hypoxia training of claim 5,
characterized in that
the flow-through container has an inlet and/or outlet,
wherein the flow-through container can be closed so that the flow of gas through the flow-through container is prevented.

7. The hypoxia device (1) for carrying out a hypoxia treatment and/or hypoxia training of claim 6,
characterized in that
the flow-through container has an inlet and/or outlet,
wherein the inlet and/or the outlet can be closed so that the flow of gas through the flow-through container is prevented.

8. The hypoxia device (1) for carrying out a hypoxia treatment and/or hypoxia training of claim 1,
characterized in that
the supply line and/or the discharge line can be closed so that the flow of gas through the flow-through container is prevented.

9. The hypoxia device (1) for carrying out a hypoxia treatment and/or hypoxia training of claim 1,
characterized in that
the automatic shut-off device (AV) is coupled to a controller (C),
wherein the automatic shut-off device (AV) can be controlled by the controller (C).

10. The hypoxia device (1) for carrying out a hypoxia treatment and/or hypoxia training according to claim 9,
characterized in that
the use of the CO₂ absorber (A) can be monitored by the controller (C).

11. The hypoxia device (1) for carrying out a hypoxia treatment and/or hypoxia training of claim 10,
characterized in that
the use of the CO₂ absorber (A) can be monitored by counting the number of hypoxia treatments.

12. The hypoxia device (1) for carrying out a hypoxia treatment and/or hypoxia training of claim 10,
characterized in that
the use of the CO₂ absorber (A) can be monitored by determining the time of use of the CO₂ absorber (A).

13. The hypoxia device (1) for carrying out a hypoxia treatment and/or a hypoxia training of claims 10 to 12,
characterized in that
the hypoxia device (1) for carrying out a hypoxia treatment comprises a flow sensor(S), wherein the use of the CO₂ absorber (A) can be monitored by determining the gas flow through the CO₂ absorber (A) by means of the flow sensor(S).

14. The hypoxia device (1) for carrying out a hypoxia treatment and/or hypoxia training of claim 10,
characterized in that
the hypoxia device (1) for carrying out a hypoxia treatment comprises a CO₂ sensor(S), wherein the monitoring of the use of the CO₂ absorber (A) can be monitored by a CO₂ sensor(S).

15. The hypoxia device (1) for carrying out a hypoxia treatment and/or hypoxia training of claim 10,
characterized in that
the automatic shut-off device (AV) comprises a valve (V) by means of which the gas supply from the gas reservoir (10) to the gas dispensing device (20) can be interrupted.

16. The hypoxia device (1) for carrying out a hypoxia treatment and/or hypoxia training of claim 10,
characterized in that
the automatic shut-off device (AV) comprises a blocking element by means of which the gas flow from the gas reservoir (10) to the gas dispensing device (20) can be interrupted by rendering the CO₂ absorber (A) unusable.

17. A method for carrying out a hypoxia treatment and/or hypoxia training, comprising the method steps of:
supplying and/or discharging a gas mixture from a gas reservoir (10) via a supply line (40) and/or discharge line (50) and a CO₂ absorber (A) arranged in the supply line (40) and/or discharge line (50) to a gas dispensing device (20),
monitoring the use of the CO₂ absorber (A),
controlling an automatic shut-off device (AV) depending on the use of the CO₂ absorber (A).

18. The method for carrying out a hypoxia treatment and/or hypoxia training of claim 17,
characterized in that
monitoring the use of the CO₂ absorber (A) comprises monitoring the consumption of the CO₂ absorber (A).

19. The method for carrying out a hypoxia treatment and/or hypoxia training of claim 17,
characterized in that
controlling the automatic shut-off device (AV) comprises controlling a blocking element.

20. The method for carrying out a hypoxia treatment and/or hypoxia training of claim 19,
characterized in that
the blocking element comprises a controllable valve and/or a sealable flow-through container.

21. The method for carrying out a hypoxia treatment and/or hypoxia training of claim 19,
characterized in that
the blocking element is controlled to interrupt and/or change the gas flow through the hypoxia device (1) for carrying out a hypoxia treatment and/or hypoxia training.

22. The method for carrying out a hypoxia treatment and/or hypoxia training of claim 19,
characterized in that
controlling the blocking element results in supplying ambient air into the hypoxia device (1) for carrying out a hypoxia treatment and/or hypoxia training.

23. The method for carrying out a hypoxia treatment and/or hypoxia training of claim 17,
characterized in that
the CO₂ absorber (A) is arranged in a flow-through container.

24. The method for carrying out a hypoxia treatment and/or hypoxia training of claim 17,
characterized in that
the CO₂ absorber (A) is arranged in a flow-through container,
wherein the flow-through container has an inlet and/or outlet,
wherein the flow-through container can be closed so that the flow of gas through the flow-through container is prevented.

25. The method for carrying out a hypoxia treatment and/or hypoxia training of claim 24,
characterized in that
the inlet and/or outlet can be closed.

26. The method for carrying out a hypoxia treatment and/or hypoxia training of claim 24,
characterized in that
the gas flow through the flow-through container is prevented by a gas-impermeable substance.

27. The method for carrying out a hypoxia treatment and/or hypoxia training of claim 17,
characterized in that
monitoring the use of the CO₂ absorber (A) is monitored by measuring the CO₂ concentration in the hypoxia device (1) for carrying out a hypoxia treatment.

28. The method for carrying out a hypoxia treatment and/or hypoxia training of claim 27,
characterized in that
monitoring the use of the CO₂ absorber (A) is carried out with a CO₂ sensor(S) by recording a measured value.

29. The method for carrying out a hypoxia treatment and/or hypoxia training of claim 27,
characterized in that
the CO₂ sensor(S) is arranged in the gas reservoir (10), the gas dispensing device (20), the supply line (40), and/or the discharge line (50).

30. The method for carrying out a hypoxia treatment and/or hypoxia training of claim 27,
characterized in that
the measurement value determined by the CO₂ sensor(S) is compared to a previously defined threshold value.

31. The method for carrying out a hypoxia treatment and/or hypoxia training of claim 30,
characterized in that
a first threshold value is fixed and/or a second threshold value is variably set.

32. The method for carrying out a hypoxia treatment and/or hypoxia training of claim 30,
characterized in that
a warning is output when the threshold value is reached and/or exceeded.

33. The method for carrying out a hypoxia treatment and/or hypoxia training of claim 32,
characterized in that
the warning is output before the blocking element is controlled to interrupt and/or change the gas flow in the hypoxia device (1) for carrying out a hypoxia treatment and/or hypoxia training.
